(19) United States
(12) Patent Application Publication
(10) Pub. No.: US 2022/0111192 A1
(43) Pub. Date: Apr. 14, 2022

(54) BODY CAVITY CONDUIT DEVICES, SYSTEMS, AND METHODS OF USE

(71) Applicant: The University of North Carolina at Chapel Hill, Chapel Hill, NC (US)

(72) Inventors: Clayton Warren COMMANDER, Chapel Hill, NC (US); Catherine Berg SREDZIENSKI, Chapel Hill, NC (US)

(73) Assignee: The University of North Carolina at Chapel Hill, Chapel Hill, NC (US)

(21) Appl. No.: 17/495,134

(22) Filed: Oct. 6, 2021

Related U.S. Application Data
(60) Provisional application No. 63/089,307, filed on Oct. 8, 2020.

(51) Int. Cl. A61M 39/02

(52) U.S. Cl. CPC A61M 39/0247; A61M 2039/027; A61M 2039/0273; A61M 2039/0291; A61M 2039/0279; A61M 2039/0297

(57) ABSTRACT

This present disclosure provides devices, systems, and methods relating to the management of a subject's medical needs involving safely accessing a body cavity. In particular, the present disclosure is directed to body cavity conduit devices and systems that facilitate repeated access to a subject's body cavity for removing fluid or performing a procedure in a manner that provides enhanced comfort, safety, and efficacy.

### CROSS REFERENCE TO RELATED APPLICATION

This application claims priority to and the benefit of U.S. Provisional Patent Application No. 63/089,307 filed Oct. 8, 2020, which is incorporated herein by reference in its entirety for all purposes.

### FIELD

This present disclosure provides devices, systems, and methods relating to the management of a subject's medical needs involving safely accessing a body cavity. In particular, the present disclosure is directed to body cavity conduit devices and systems that facilitate repeated access to a subject's body cavity for removing fluid or performing a procedure in a manner that provides enhanced comfort, safety, and efficacy.

### BACKGROUND

The ability to repeatedly access a patient's body cavity to, for example, drain a bodily fluid or perform a medical procedure, presents an important challenge to the medical community. Although there are many devices currently available that facilitate access to a body cavity of a patient, they generally overlook many important considerations from the perspective of the patient, including providing a safe, effective, and comfortable means for managing their particular medical needs.

For example, in the urological setting, there are generally two common reasons that people cannot void urine normally or experience urinary retention. These are neurogenic bladder and bladder outlet obstruction. Neurogenic bladder is a common condition resulting from nerve and/or muscle dysfunction of the bladder. This is commonly seen as a complication of diabetes mellitus, spinal cord injury, pelvic surgery, brain tumors, Parkinson's disease, stroke, Multiple Sclerosis, ALS, and congenital disorders such as spina bifida. Additionally, bladder outlet obstruction can be caused by urethral stricture or injury, but is often caused by benign prostatic hyperplasia (BPH). Since the urethra in males is surrounded by the prostate gland, enlargement of the prostate can result in compression of the urethra leading to urinary retention or obstruction, weak stream, and urge incontinence. These and other symptoms are often referred to in the literature as LUTS (lower urinary tract symptoms).

BPH is among the most common clinical conditions affecting older men and it progresses with age. BPH is present in approximately 50% of men ages 50-60 and 90% of men over age 80. It is estimated that LUTS can be present in up to 70% of men over the age of 60, and up to 90% of men 90 or older. Current treatments range from lifestyle modifications to medications to surgical and minimally invasive options, but some patients ultimately have to resort to bladder drainage. According to US Census population prediction models, by the year 2030, all baby boomers will have reached the age of 65 or older. Also, by 2035, the number of people in the US over the age of 85 will nearly double to almost 12 million and will triple by 2060 to 19 million. Other patients that may benefit from alternate urinary drainage include those with urinary incontinence, chronic urinary retention, pelvic surgery requiring temporary urine diversion and also palliative care needs.

Patients who are unable to void urine from their urethra have limited minimally invasive options for bladder drainage. Some perform intermittent self catheterization (ISC) via the urethra, and some have a continuous indwelling transurethral bladder catheter, or “Foley” catheter. Frequent or long term transurethral drainage can cause pain, discomfort, frequent infections, and tissue breakdown from repeated trauma to the tissue. Patients desiring a long-term minimally invasive solution typically undergo suprapubic (SP) cystostomy placement by an interventional radiologist or urologist. Using local anesthesia and moderate sedation, a catheter is placed percutaneously (through the skin) above the pubic bone into the bladder. The distal aspect of the catheter is surrounded by a retention balloon which is inflated with sterile water after insertion into the bladder which prevents dislodgement of the catheter. Any patient unable to spontaneously void urine from the urethra is a potential candidate for suprapubic cystostomy placement. If the bladder is able to safely store urine, an intermittent option for drainage should be considered.

There are several problems with current indwelling percutaneous SP catheters due to the length, profile, and physical characteristics of the catheters currently in use. Conventional suprapubic cystostomy catheters are approximately 40 cm in length and extend from the skin entry site approximately 30-35 cm. They are difficult to secure and often get pulled and tugged, leading to ostomy tract trauma, stretching, leakage, and need for upsizing of the catheter. In addition, they are often dislodged (often with the retention balloon inflated) which traumatizes the tract. Replacement is urgent as the patient has no other way of emptying the bladder so weekend/late night ER visits to have the catheter replaced are common, causing patient stress, increased cost and strain on the healthcare system. Conventional SP catheters are difficult to conceal under clothing and make ambulation difficult. Many patients leave the catheter connected to a drainage bag for continuous bladder emptying; however, some patients choose to “cap” the end of the catheter and perform intermittent bladder drainage.

In addition, there is often a problem with the balloon-tissue interface at the bladder surface because it is difficult to maintain the catheter in a stable position. To avoid leakage of urine at the site of insertion, the balloon should have good apposition against the bladder wall at all times. Too much pressure, and tissue damage can occur. Too little pressure, and a good seal is not achieved. Patient movement, a lack of standardized securing methods, and different body habitus scenarios all contribute to leakage and catheter instability. Furthermore, with a long, indwelling catheter, the risk of infection is always present due to the same catheter being in place for extended periods of time. Given their length, the catheters can become clogged with sediments which precipitate from the urine after lying static in the catheter. Traditional SP catheters have to be changed every 1-3 months due to these limitations. The British Association of Urological Surgeons (BAUS) acknowledges that limitations such as recurrent infections and repeated catheter failure due to blockage can cause a burden for patients and clinical resources and research is needed in this area.

These and other limitations are also present in other contexts, outside the urological setting. Therefore, there is a need to provide a better, long-term solution that has positive outcomes for self-care, fewer complications, longer device life, and better quality of life for this expanding patient population.

### SUMMARY

Embodiments of the present disclosure include a body cavity conduit device. In accordance with these embodiments, the device includes a proximal end comprising a low-profile first opening positioned external to a subject's body, a distal end comprising a second opening positioned within a body cavity of the subject, and a stem comprising a channel that connects the proximal end and the distal end. In some embodiments, either the proximal end and/or the distal end comprises a self-sealing valve.

In some embodiments, the proximal end comprises a self-sealing valve, which can vary in its relative position within the stem channel In some embodiments, the distal end comprises a self-sealing valve, which can vary in its relative position within the stem channel. In some embodiments, the self-sealing valve(s) is/are configured to prevent fluid flow through the stem channel.

In some embodiments, the self-sealing valve comprises a perforation for insertion of an accessory component. In some embodiments, the perforation is configured to form a seal around the accessory component when inserted into the stem channel.

In some embodiments, the proximal end comprises a cover cap and plug for insertion into the first opening. In some embodiments, the plug is configured to form a seal when inserted into the first opening. The devices of the present disclosure can be utilized with or without the proximal end cover cap in place.

In some embodiments, the low-profile first opening does not extend outwardly more than 5 cm from the subject's body. In some embodiments, the low-profile first opening extends outwardly from about 1 cm to about 3 cm from the subject's body.

In some embodiments, the proximal end further comprises a retention balloon port and a retention balloon valve. In some embodiments, the distal end comprises a retention balloon that is positioned within the subject's body cavity.

In some embodiments, the stem comprises a retention balloon channel extending from the retention balloon port to the retention balloon, wherein the retention balloon channel is parallel to the stem channel. The retention balloon can be positioned at various locations along the distal end of the device. In some embodiments, the retention balloon is positioned such that the distal end of the stem channel extends past the inflated balloon and into the body cavity (e.g., from 0 mm to about 5 mm past the inflated balloon and into the body cavity).

In some embodiments, the retention balloon is configured to hold from about 2 ml to about 25 ml of a fluid.

In some embodiments, the stem is no greater than 30 cm in length. In some embodiments, the stem is from about 1 cm to about 15 cm in length. In some embodiments, the stem is no greater than 35 Fr in diameter. In some embodiments, the stem is from about 10 Fr to about 30 Fr in diameter.

In some embodiments, the accessory component comprises at least one of a bladder catheter, a gastrostomy tube, a drainage catheter, an ablation catheter, a catheter for instilling medication or fluid, an endoscope, a needle, and/or a biopsy tool.

In some embodiments, the subject's body cavity comprises at least a portion of a bladder cavity, a stomach cavity, an intestinal cavity, a peritoneal cavity, a pleural cavity, a fluid collection cavity, a retroperitoneal cavity, a kidney cavity or a gall bladder.

Embodiments of the present disclosure also include a kit. In accordance with these embodiments, the kit includes any of the body cavity conduit devices described herein, and an obturator tool, device, balloon, and/or dilator.

In some embodiments, the kit further comprises at least one of the following: a guidewire, a dilator, a stem measuring device, a slip tip syringe, bladder catheter, a gastrostomy tube, a drainage catheter, an ablation catheter, a catheter for instilling medication or fluid, an endoscope, a needle, a biopsy tool, a splitable or separating removable sheath, and a cleaning tool for the channel.

Embodiments of the present disclosure also include a method for inserting any of the body cavity conduit devices described herein into a body cavity of a subject. In accordance with these embodiments, the method includes inserting an obturator tool, device, balloon, and/or dilator positioned at the distal end of the device over a guidewire and into the subject's body cavity such that the distal end of the device is positioned in the subject's body cavity and the proximal end of the device is positioned external to the subject's body, inflating the retention balloon with a suitable volume of a fluid to secure placement of the device in the subject, and removing the obturator and the guidewire.

### BRIEF DESCRIPTION OF THE DRAWINGS

FIGS. 1A-1B include representative side views of a body cavity conduit device of the present disclosure with the retention balloon inflated (FIG. 1A), reflecting a secured position within a subject's body cavity, and with the retention balloon deflated (FIG. 1B), reflecting a unsecured position within a subject's body cavity.

FIGS. 2A-2C include a representative side view of the body cavity conduit device with the retention balloon inflated (FIG. 2A), a representative perspective view of the body cavity conduit device with the retention balloon inflated (FIG. 2B), and a top view of the body cavity conduit device with the retention balloon inflated (FIG. 2C). FIGS. 2A and 2C provide exemplary dimensions of a length of the device and a width of the device, according to one embodiment of the present disclosure.

FIGS. 3A-3B include representative cross-sectional views of a body cavity conduit device of the present disclosure showing a cover cap and plug that can be inserted into the proximal opening of the device when not in use (FIG. 3A) or left open when in use (FIG. 3B).

FIGS. 4A-4B include representative cross-sectional views of the proximal end of a body cavity conduit device of the present disclosure. A cross-sectional side view shows the proximal end of the device with the plug of the cover cap inserted into the proximal opening (FIG. 4A), reflecting the position of the device when not in use, and a cross-sectional perspective view of the device without the plug inserted (FIG. 4B), reflecting the position of the device when not in use.

FIG. 5 includes a representative cross-sectional view of the distal end of a body cavity conduit device of the present disclosure showing a self-sealing valve.

FIG. 6 includes a representative cross-sectional view of a body cavity conduit device of the present disclosure positioned within a body cavity of a subject (e.g., bladder) with a cover cap in the “open” position to accept an accessory component (e.g., drainage catheter to provide drainage of fluid from the body cavity).

FIG. 7 includes a representative cross-sectional view of a body cavity conduit device of the present disclosure positioned within a body cavity of a subject (e.g., bladder) with the cover cap closed.

FIG. 8 includes a representative cross-sectional view of a body cavity conduit device of the present disclosure positioned within a body cavity of a subject (e.g., bladder) with the cover cap closed, illustrating the low-profile design of the device, as compared to a conventional suprapubic catheter.

### DETAILED DESCRIPTION

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human) In some embodiments, the subject may be a human or a non-human. In one embodiment, the subject is a human The subject or patient may be undergoing various forms of treatment.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

“Therapy” and/or “therapy regimen” generally refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. In some embodiments, the treatment comprises the treatment, alleviation, and/or lessening of pain.

Unless otherwise defined herein, scientific and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Conduit Devices

Embodiments of the present disclosure include a body cavity conduit device having a low-profile design that provides a stable access point for repeated intermittent or continuous use for removing fluid or performing a procedure in a manner that provides enhanced comfort, safety, and efficacy. Although the body conduit devices of the present disclosure can be used to facilitate access to any body cavity of a subject and for any purpose, some exemplary embodiments of the present disclosure include use of the body conduit devices and systems described herein as a low-profile suprapubic cystostomy conduit device (e.g., for drainage of urine from the bladder). However, as would be readily understood by one of ordinary skill in the art based on the present disclosure, the body cavity conduit devices and systems described herein can provide a safe, comfortable, and effective access point for performing a variety of medical procedures.

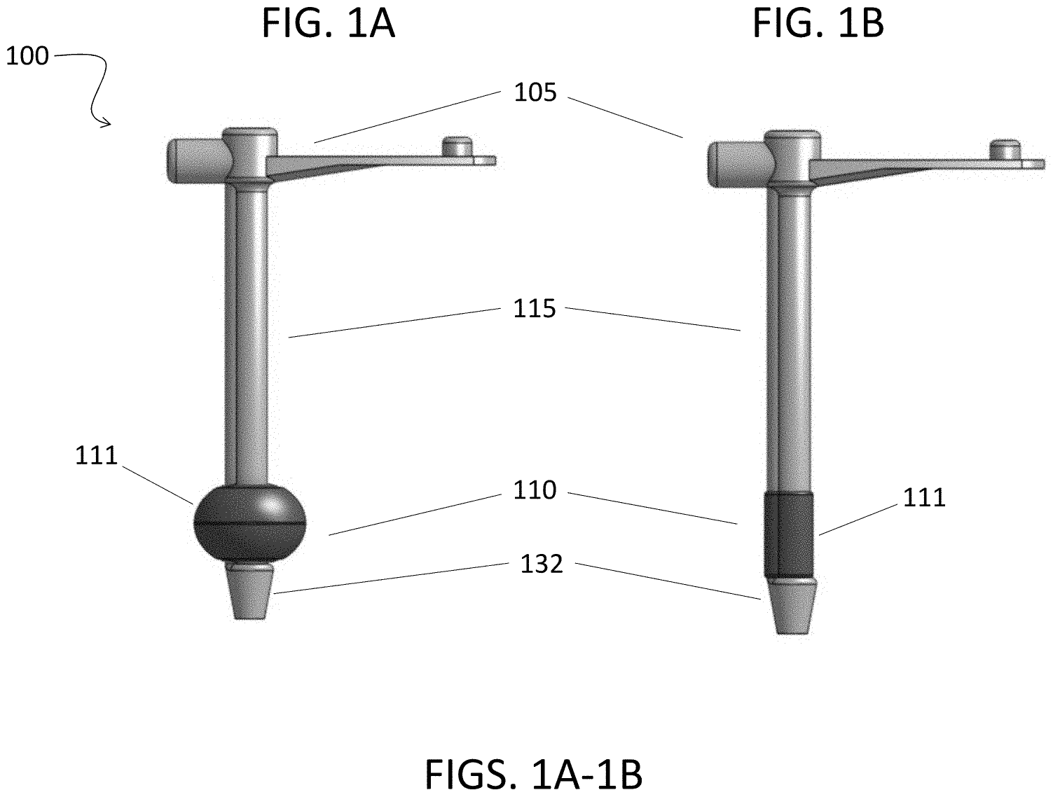

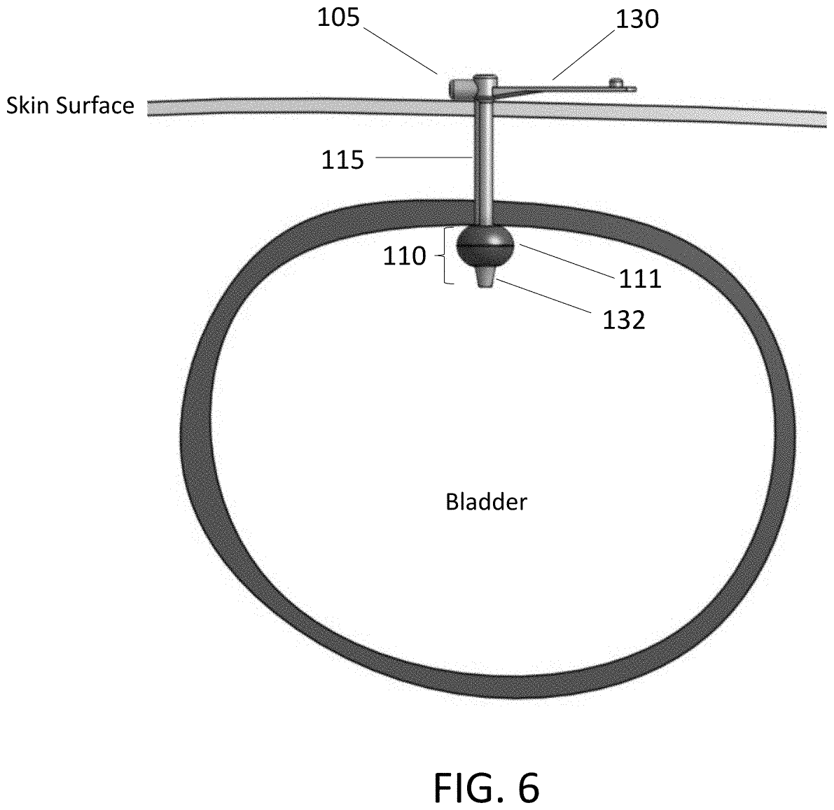

In accordance with these embodiments, and as shown in FIGS. 1A-1B, body cavity conduit devices of the present disclosure (100) include a proximal end (105), a distal end (110), and a stem (115) that connects the proximal end and the distal end. The stem includes an inner channel that provides, for example, a pathway for fluid to flow in or out of a subject's body cavity or for an accessory medical component to enter a subject's body cavity to perform a desired medical procedure. The proximal end (105) corresponds to the end of the device that is outward facing. That is, when the device (100) is secured inside a subject's body, the proximal end (105) is positioned external to a subject's body and is readily accessible by the subject or by a medical professional (see also FIGS. 6-8). In contrast to many currently available conduit devices, the proximal end (105) of the body cavity conduit devices of the present disclosure has a significantly lower profile design to maximize patient comfort without sacrificing safety or efficacy. The proximal end (105) also provides an initial point of entry into a subject's body cavity for an accessory component (e.g., catheter). The distal end (110) corresponds to the end of the device that is inward facing. That is, when the device (100) is secured inside a subject's body, the distal end (110) is positioned within a body cavity of a subject (e.g., bladder). The distal end (110) also includes a retention balloon (111) that, when inflated (FIG. 1A) helps secure the device (100) in position. When deflated (FIG. 1B), the retention balloon (111) is compressed around the distal end (110) of the device such that the device can be inserted or removed. The distal end (110) is generally tapered, as shown in FIG. 1B).

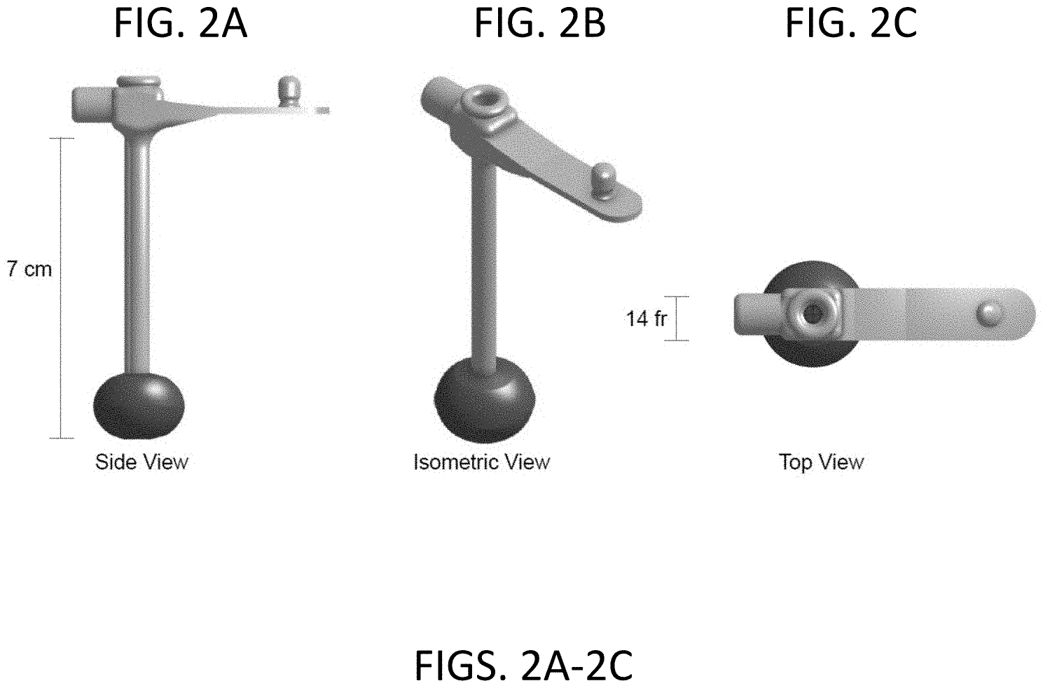

As shown in FIGS. 2A-2C, body cavity conduit devices of the present disclosure (100) can be various sizes and dimensions. The particular size and dimensions of the device will depend on various factors, including but not limited to, the size of the subject, the location of the subject's body cavity, the primary medical need being addressed with the device, and the like. In some embodiments, the portion of the device (100) that is positioned inside a subject's body, which includes the stem (115) and the distal end (110), can be about 7 cm in length (FIG. 2A), while the width of the proximal end (105) can be about 14 Fr (FIG. 2C).

Although FIGS. 2A-2C provide exemplary embodiments pertaining to, for example, use of the device (100) as a suprapubic cystostomy device, other sizes and dimensions can be used that are suitable for accessing other body cavities and performing other medical procedures. That is, the body cavity conduit devices of the present disclosure (100) can be adapted to any subject for any relevant procedure, for example, by simply altering the length of the stem (115), but maintaining the low-profile design of the proximal end (105) of the device. In some embodiments, the stem (115) is not greater than about 30 cm in length. In some embodiments, the stem (115) is not greater than about 25 cm in length. In some embodiments, the stem (115) is not greater than about 20 cm in length. In some embodiments, the stem (115) is not greater than about 15 cm in length. In some embodiments, the stem (115) is not greater than about 10 cm in length. In some embodiments, the stem (115) is not greater than about 5 cm in length. In some embodiments, the stem (115) is from about 1 cm to about 30 cm in length. In some embodiments, the stem (115) is from about 5 cm to about 30 cm in length. In some embodiments, the stem (115) is from about 5 cm to about 25 cm in length. In some embodiments, the stem (115) is from about 5 cm to about 20 cm in length. In some embodiments, the stem (115) is from about 5 cm to about 15 cm in length. In some embodiments, the stem (115) is from about 5 cm to about 10 cm in length. In some embodiments, the stem (115) is from about 10 cm to about 30 cm in length. In some embodiments, the stem (115) is from about 10 cm to about 25 cm in length. In some embodiments, the stem (115) is from about 10 cm to about 20 cm in length. In some embodiments, the stem (115) is from about 10 cm to about 15 cm in length. In some embodiments, the stem (115) is from about 15 cm to about 30 cm in length. In some embodiments, the stem (115) is from about 15 cm to about 25 cm in length. In some embodiments, the stem (115) is from about 15 cm to about 20 cm in length.

To achieve a low-profile design as described above, the proximal end (105) of the device (100) can be configured such that it does not extend significantly beyond the outer surface of a subject's body (e.g., skin surface; see FIGS. 6-8). In some embodiments, the proximal end (105) of the device (100) includes a low-profile first opening that does not extend outwardly more than 5 cm from the subject's body. In some embodiments, the proximal end of the device does not extend outwardly more than 4 cm from the subject's body. In some embodiments, the proximal end of the device does not extend outwardly more than 3 cm from the subject's body. In some embodiments, the proximal end of the device does not extend outwardly more than 2 cm from the subject's body. In some embodiments, the proximal end of the device does not extend outwardly more than 1 cm from the subject's body. In some embodiments, the low-profile first opening of the proximal end of the device extends outwardly from about 1 cm to about 5 cm from the subject's body. In some embodiments, the low-profile first opening of the proximal end of the device extends outwardly from about 1 cm to about 4 cm from the subject's body. In some embodiments, the low-profile first opening of the proximal end of the device extends outwardly from about 1 cm to about 3 cm from the subject's body. In some embodiments, the low-profile first opening of the proximal end of the device extends outwardly from about 1 cm to about 2 cm from the subject's body. In some embodiments, the low-profile first opening of the proximal end of the device extends outwardly from about 2 cm to about 5 cm from the subject's body. In some embodiments, the low-profile first opening of the proximal end of the device extends outwardly from about 3 cm to about 5 cm from the subject's body. In some embodiments, the low-profile first opening of the proximal end of the device extends outwardly from about 4 cm to about 5 cm from the subject's body. In some embodiments, the low-profile first opening of the proximal end of the device extends outwardly from about 2 cm to about 4 cm from the subject's body. In some embodiments, the low-profile first opening of the proximal end of the device extends outwardly from about 2 cm to about 3 cm from the subject's body. In some embodiments, the low-profile first opening of the proximal end of the device extends outwardly from about 3 cm to about 5 cm from the subject's body. In some embodiments, the low-profile first opening of the proximal end of the device extends outwardly from about 4 cm to about 5 cm from the subject's body.

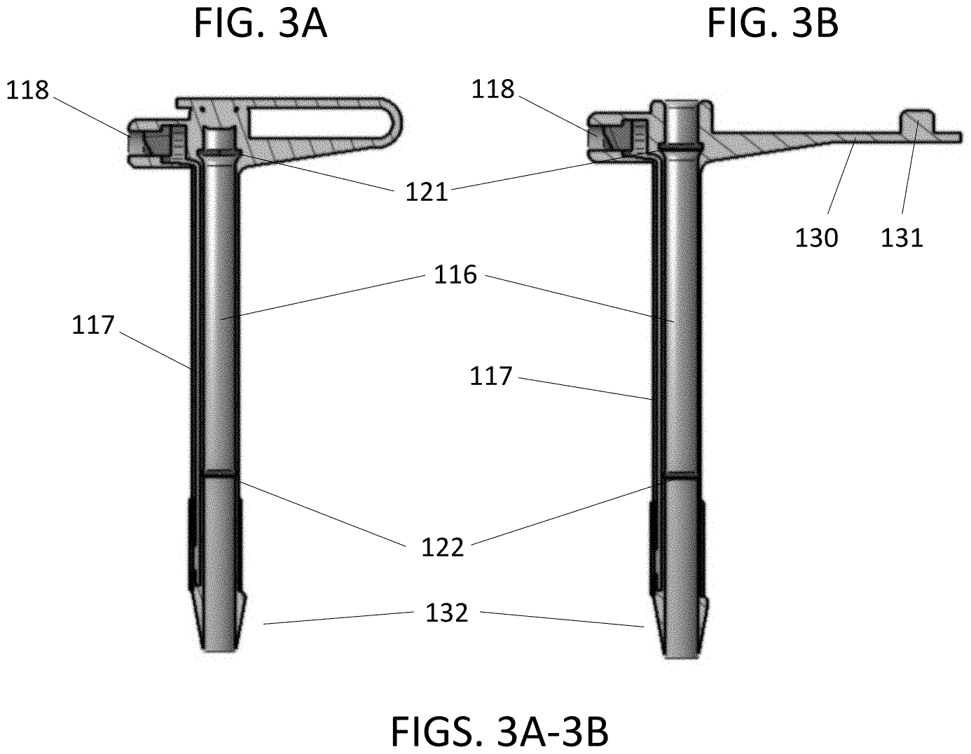

As described further herein, the stem (115) comprises a channel (116) through which fluid can flow (e.g., drain urine from the bladder) and/or through which a medical accessory component can be inserted. As shown in FIGS. 3A-3B and FIGS. 6-8, the stem (115) includes a channel (116) that can be various diameters, depending on the specific use of the body cavity conduit device, among other factors. In some embodiments, the stem is no greater than about 35 Fr in diameter. In some embodiments, the stem is no greater than about 30 Fr in diameter. In some embodiments, the stem is no greater than about 25 Fr in diameter. In some embodiments, the stem is no greater than about 20 Fr in diameter. In some embodiments, the stem is no greater than about 15 Fr in diameter. In some embodiments, the stem is no greater than about 10 Fr in diameter. In some embodiments, the stem is from about 10 Fr to about 50 Fr in diameter. In some embodiments, the stem is from about 10 Fr to about 40 Fr in diameter. In some embodiments, the stem is from about 10 Fr to about 30 Fr in diameter. In some embodiments, the stem is from about 20 Fr to about 50 Fr in diameter. In some embodiments, the stem is from about 30 Fr to about 50 Fr in diameter. In some embodiments, the stem is from about 30 Fr to about 40 Fr in diameter.

In some embodiments, the stem (115) further comprises a retention balloon channel (117), as shown in FIGS. 3A-3B, that extends from a retention balloon port (118) at the proximal end of the device to the retention balloon (111) at the distal end of the device. The retention balloon channel (117) is generally parallel to the stem channel (116) and provides a pathway for which fluid (e.g., saline solution or sterile water) can be passed to inflate the retention balloon (111) from outside the subject's body. In some embodiments, the distal end of the stem channel (132) extends past the inflated retention balloon (111) and into the body cavity. In some embodiments, the distal end of the stem channel (132) extends from 0 mm to about 5 mm past the inflated retention balloon (116) (see, e.g., FIGS. 1A-1B, 3A-3B, and 5-8).

In some embodiments, the retention balloon is configured to hold certain amounts of a fluid in order to securely position the body conduit device within the subject. In some embodiments, the retention balloon is configured to hold about 2 ml to about 25 ml of a fluid. In some embodiments, the retention balloon is configured to hold about 2 ml to about 20 ml of a fluid. In some embodiments, the retention balloon is configured to hold about 2 ml to about 15 ml of a fluid. In some embodiments, the retention balloon is configured to hold about 2 ml to about 10 ml of a fluid. In some embodiments, the retention balloon is configured to hold about 2 ml to about 5 ml of a fluid. In some embodiments, the retention balloon is configured to hold about 5 ml to about 25 ml of a fluid. In some embodiments, the retention balloon is configured to hold about 10 ml to about 25 ml of a fluid. In some embodiments, the retention balloon is configured to hold about 15 ml to about 25 ml of a fluid. In some embodiments, the retention balloon is configured to hold about 20 ml to about 25 ml of a fluid. In some embodiments, the retention balloon is configured to hold about 5 ml to about 20 ml of a fluid. In some embodiments, the retention balloon is configured to hold about 10 ml to about 20 ml of a fluid. In some embodiments, the retention balloon is configured to hold about 10 ml to about 15 ml of a fluid.

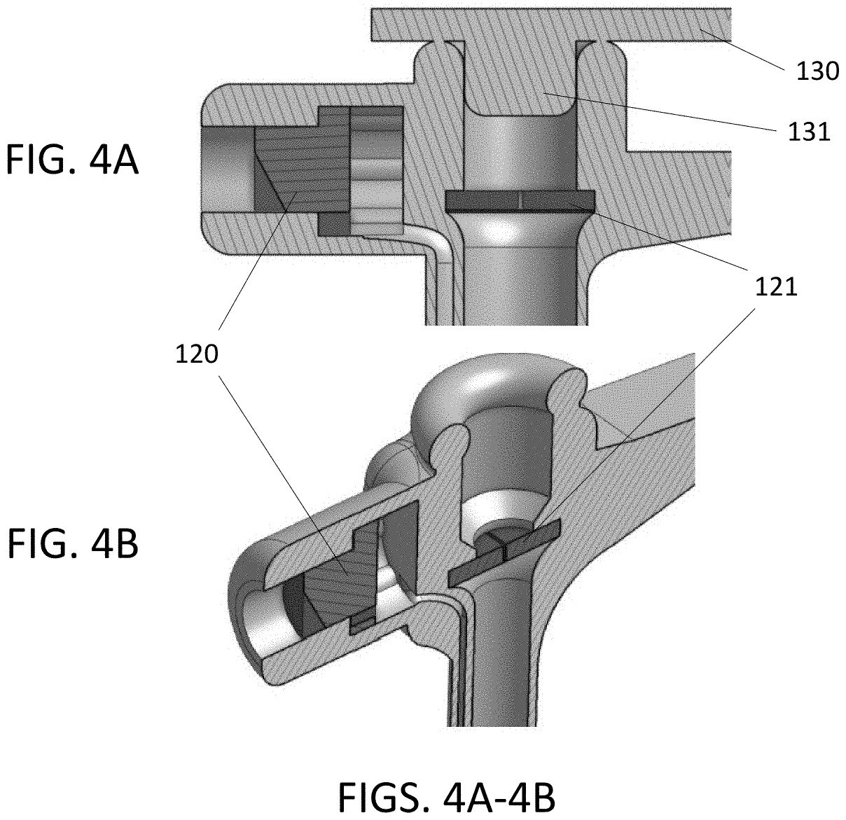

As shown in FIGS. 3A-3B and FIGS. 4A-4B, the body cavity conduit devices of the present disclosure can include one or more valves that can be configured to manage fluid flow into or out of the device, and/or to control the inflation/deflation of the retention balloon. In some embodiments, the proximal end (105) of the device includes retention balloon valve (120) that is contained within the retention balloon port (118). The retention balloon valve (120) provides a means for controlling the amount of fluid (e.g., saline solution or sterile water) that enters and exits the retention balloon (111) through the retention balloon channel (117).

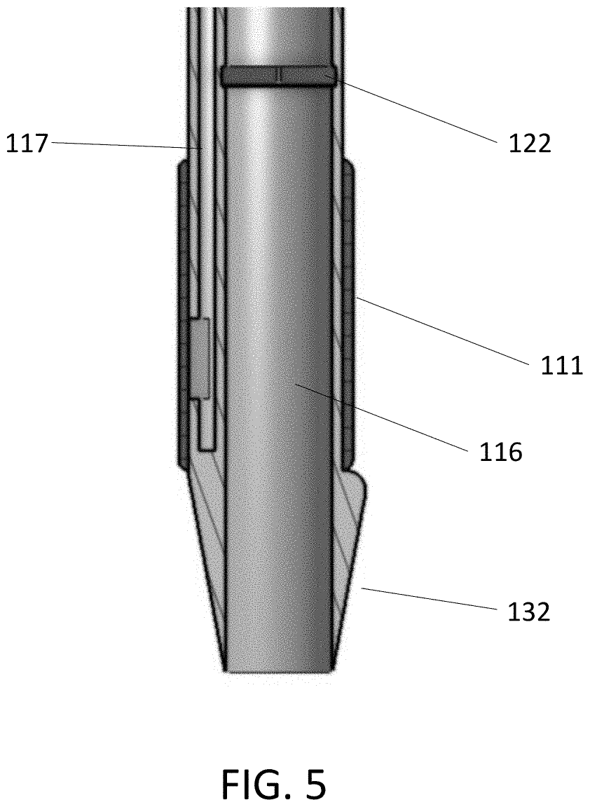

Additionally, as shown in FIGS. 3A-3B and FIGS. 4A-4B, the body cavity conduit devices of the present disclosure can include one or more valves at the proximal end (105) and/or the distal end (110) of the device. In some embodiments, the proximal end (105) includes a valve (121) that is positioned just underneath the first opening in the proximal end, adjacent to the retention balloon port (118), as shown in FIGS. 4A-4B. In some embodiments, the distal end (110) includes a valve (122) that is positioned just above the retention balloon (122), as shown in FIG. 5 and FIGS. 4A-4B. In some embodiments, the body conduit devices of the present disclosure include a valve at the proximal end (105) of the device, but not at the distal end (110) of the device. In some embodiments, the body conduit devices of the present disclosure include a valve at the distal end (110) of the device, but not at the proximal end (105) of the device. In some embodiments, the body conduit devices of the present disclosure include a valve at the proximal end (105) of the device and at the distal end (110) of the device. In some embodiments, the body conduit devices of the present disclosure include a valve at the proximal end (105) of the device, at the distal end (110) of the device, and at at least one more position within the device. Any of the self-sealing valves can be positioned in various locations within the stem channel of the device.

In accordance with these embodiments, the valves at the proximal and distal ends of the device (121 and 122), can be configured to be self-sealing. Self-sealing can be achieved by various mechanisms, provided that the valves (121 and 122) can prevent fluid flow through the stem channel (116) when not in use (e.g., urine drainage from a bladder), and/or prevent fluid flow through the stem channel (116) when an accessory component is inserted through the channel to access a subject's body cavity. In some embodiments, the self-sealing valves (121 and 122) include a perforation that allows for insertion of an accessory component (e.g., catheter) through the valve, such that the integrity of the valve and its ability to seal off fluid remains intact. As would be recognized by one of ordinary skill in the art based on the present disclosure, valves can be made out of various materials and can have various other configurations, provided they are self-sealing. For example, the valves can be made of a silicone-based material, or another flexible, biocompatible material. Valves can also be configured to have perforations comprising any size or shape provided that the function of the valve remains intact.

In some embodiments, the body cavity conduit devices of the present disclosure also include a cover cap (130) and plug (131) at the proximal end (105) of the device, as shown in FIGS. 1-4. For example, as shown in FIG. 3B and FIG. 4A, the proximal end (105) of the device can include a cover cap (130) that extends in a continuous manner from a position on the proximal end of the device, such that it can be bent back over the opening of the proximal end to provide a sealing mechanism via insertion of the plug (131) into the opening. Though not necessary for the body conduit device of the present disclosure to achieve its intended purpose, the cover cap (130) and plug (131) can be included in the device in order to provide additional comfort to the subject when the device is not in use (e.g., secondary mechanism for preventing fluid flow). As shown, the cover cap (130) and plug (131) are configured in a manner so as not to significantly add to the size of the proximal end of the device in order to maintain the low-profile design.

### 3. Systems and Methods

Embodiments of the present disclosure also include systems and kits for accessing a body cavity in a subject using the body cavity conduit devices described herein in order to remove a fluid from the cavity and/or to perform a medical procedure. For example, in some embodiments, the body cavity conduit devices of the present disclosure can be used in conjunction with one or more accessory components. In some embodiments, the accessory component includes, but is not limited to a bladder catheter, a gastrostomy tube, a drainage catheter, an ablation catheter, a catheter for instilling medication or fluid, an endoscope, a needle, and/or a biopsy tool. In accordance with these embodiments, a subject's body cavity that can be accessed using the devices and systems of the present disclosure includes, but is not limited to, a bladder cavity, a stomach cavity, an intestinal cavity, a peritoneal cavity, a pleural cavity, a fluid collection cavity, a retroperitoneal cavity, a kidney cavity, or a gall bladder.

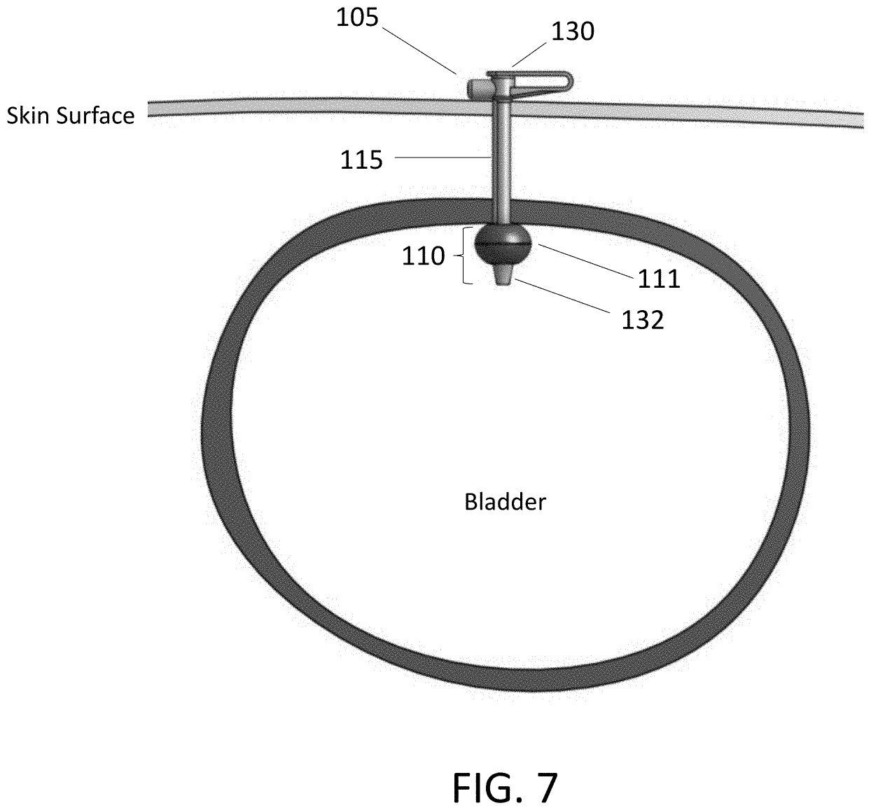

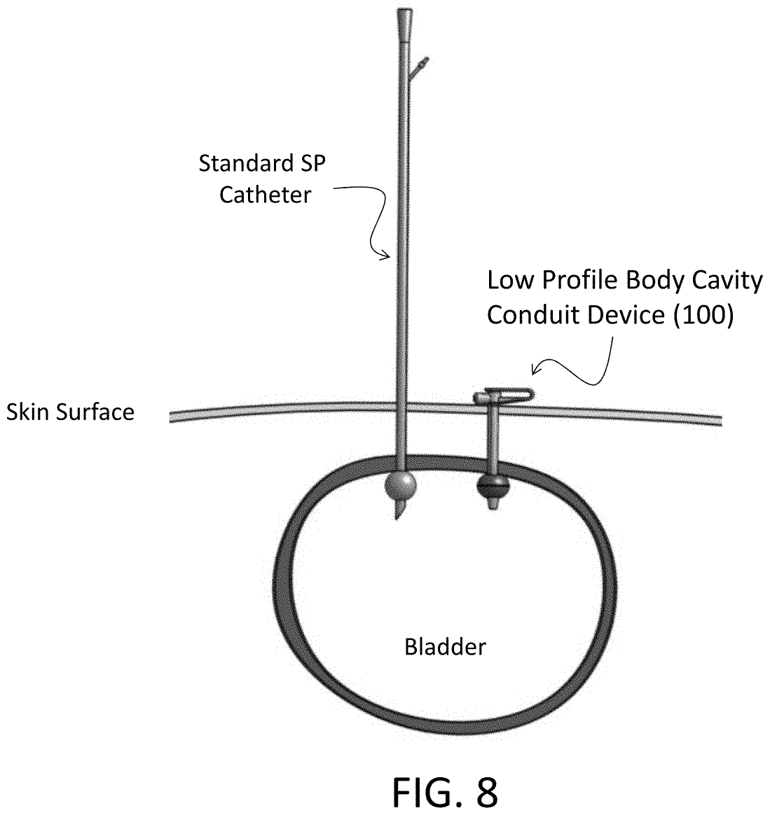

For example, as shown in FIG. 6, a body cavity conduit device of the present disclosure can be used to provide access to the bladder of a subject using a urinary catheter. When not in use, as shown in FIG. 7, the body cavity conduit device can be sealed with a cover cap and plug, while maintain a low-profile design that maximizes patient comfort and reduces the negative impact on patient lifestyle. As shown in FIG. 8, the body cavity conduit devices and systems of the present disclosure offer this, and many other improvements compared to currently available drainage devices.

Embodiments of the present disclosure also include a kit. In accordance with these embodiments, the kit includes any of the body cavity conduit devices described herein, and an obturator tool, device, balloon, and/or dilator. In some embodiments, the kit also includes a guidewire, a dilator, a stem measuring device, a slip tip syringe, bladder catheter, a gastrostomy tube, a drainage catheter, an ablation catheter, a catheter for instilling medication or fluid, an endoscope, a needle, a biopsy tool, a splitable or separating removable sheath, and a cleaning tool for the channel. These additional components of the kit can be used with the body cavity conduit devices of the present disclosure to, for example, drain a fluid from a body cavity of a subject, and/or to perform a medical procedure. In some embodiments, the additional components of the kits provided herein are useful for inserting and/or removing the body conduit devices from a subject. In some embodiments, the kit further includes instructions for accessing a body cavity of a subject, for draining a fluid from a body cavity of a subject, and for inserting/removing a body conduit device from a subject.

For example, embodiments of the present disclosure also include a method for inserting any of the body cavity conduit devices described herein into a body cavity of a subject. In accordance with these embodiments, the method includes inserting an obturator tool, device, balloon, and/or dilator positioned at the distal end of the device over a guidewire and into the subject's body cavity such that the distal end of the device is positioned in the subject's body cavity and the proximal end of the device is positioned external to the subject's body, inflating the retention balloon with a suitable volume of a fluid to secure placement of the device in the subject, and removing the obturator and the guidewire.

### 4. Suprapubic Cystostomy (SP) Conduit Device

In some embodiments, and in particular with respect to use of the body conduit device of the present disclosure as a suprapubic cystostomy (SP) device, the device can have a profile similar to the low-profile design of a gastrostomy tube (or G-tubes). In some cases, the device is comprised of an internal retention balloon and external portion that extends approximately 1-1.5 cm above the skin entry site. Silicone or other flexible biocompatible material can be used to make the device. The device can include an external portion containing a cover cap and a balloon port for filling the retention balloon. The device can also include a proximal self-sealing valve composed of silicone or similar flexible, pliant material for entry to the open channel of the device. The device also comprises a stem (or stoma) that corresponds to the portion of the device between the skin surface and the retention balloon. The device includes a small channel extending down the stem portion of the device from the balloon fill port to the balloon which sits in the bladder.

The retention balloon can have a spherical or an oblate spheroid shape or any combination or variation thereof. The retention balloon can have a fill volume of approximately 2-25 ml of sterile water or sterile saline. The device can also include a distal self-sealing valve located at the distal portion of the device, or at any point in the stem channel that is distal to the proximal self-sealing valve, and which serves as the entry point into the bladder.

This design provides a stable balloon-tissue interface with the bladder as well as stable device-tissue interface at the skin surface. Because this is essentially a “fixed” position, there is less chance of leakage of urine from the insertion site. In addition, patients may ambulate freely and the device can work with a number of body habitus challenges because the device moves easily and in tandem with the patient. The low profile nature of the device greatly decreases accidental dislodgement and allows for better concealment of the device when not in use.

The SP conduit device can be available in a variety of standard sizes, and size selection for each patient can be determined with a standard measuring device known to those of ordinary skill in the art. In some cases, french size for width of the SP device can range from 14-30 Fr to allow for catheter sizes of 12-16 Fr, for example. Stoma size (stem channel size) for the length of device can range from about 1 cm to about 7 cm, for example.

Insertion of the SP device can occur with image guidance (e.g., using the Seldinger technique). In some cases, a local anesthetic agent and moderate sedation can be utilized during an insertion procedure. In some cases, the device can be placed via an established mature tract from an existing SP catheter. In other cases, such as for de novo placement, the distended bladder is identified with ultrasound guidance, a needle is then inserted into the bladder and confirmation of urine return, and a guidewire is placed through the needle. Confirmation of guidewire placement can be confirmed with fluoroscopy. The needle is removed and the tract serially dilated (if necessary) to desired size using dilators or balloons over the wire. A measuring device can be utilized to determine the size needed. The appropriately sized device utilizing an obturator tool, device, balloon, or dilator through the distal tip is then placed over the guidewire into the bladder. Contrast injection into the bladder, ultrasound imaging, and/or urine output confirms position. The retention balloon is then inflated to the required volume to secure the device, and the guidewire is removed; the obturator, device, balloon, and/or dilator can also be removed. This creates a small, manageable, flexible and stable access point to the bladder through which the patient or caregiver can insert a standard commercially available drainage catheter intermittently to drain the bladder. Alternatively, a standard drainage catheter can be placed through the device and secured with tape in the same fashion that a traditional SP catheter is secured.

As described further herein, the SP device can include a two-valve design that includes a proximal valve that serves as entry point to the open channel and a distal valve that is the entry point to the bladder. In some cases, the SP device is designed and construction such that presence of the proximal valve eliminates the need for a cover cap over the device. To use the device, the patient wipes the valve with an antiseptic wipe that comes in the catheter kit, for example, and then inserts a commercially available catheter. Patients who intermittently self-catheterize can do the same prior to catheterization except they are cleansing the external portion of the urethra instead. This increases usability of the device in that the patient does not need fine motor skills to manipulate a cap or “plug” in order to use the device. It eliminates a step in the drainage process. Furthermore, caps that are used in similar low profile G-tube devices often break after repeated use or the tether portion tears off and this causes the patient stress. In fact, many patients have these devices replaced simply because the cap has malfunctioned. However, a cap could be present if the patient feels more secure having the entry point covered at all times. But the presence of the proximal valve allows the device to be used equally, with or without the cap. Because the proximal and distal valves are self-sealing, there should be no leakage of urine from the device and the bladder essentially remains sealed off in between drainage sessions, whether or not a cover cap is present.

As described further herein, the valve can be designed to have a variety of configurations. For example, a slit type valve similar to that on sheaths with hemostatic valves can be used. In some cases, a round valve with a center hole that is composed of a very flexible material can be used, such that it can expand and seal around the drainage catheter and then close again when the catheter is removed. In some cases, a valve with multiple “petals” that fold in on each other can be used. A slit type valve (e.g., valve with perforations) can be used with variable “zones” in a circular shape so that the center portion is slightly more pliable to allow passage of a catheter and the outer portion is stiffer to ensure a good seal when the drainage catheter is in place, especially if continuous drainage is desired.

In some embodiments, the SP devices of the present disclosure reflect that entry into the device and use of the device occurs via the two self-sealing valves and do not include any locking mechanism or special attachment pieces, extensions, adapters, or connectors. Therefore, any off-the-shelf, size-appropriate urinary drainage catheter is compatible with the device. This greatly reduces cost, and improves patient experience for obtaining drainage supplies and the overall simplicity of the drainage process. For example, to perform bladder drainage, the patient inserts a single use drainage catheter, drains the urine, and then removes the catheter when drainage is complete.

The non-locking, open-channel aspects of the SP devices of the present disclosure offer many other advantages. Because a new drainage catheter is used at each drainage session, there is less risk of infection compared to a long term indwelling SP catheter. (Patients who intermittently self catheterize via the urethra also use a new catheter each time and often order these by the box). Because a new drainage catheter is used at each drainage session, there is minimal risk of clogging complications seen with conventional SP catheters. Additionally, there is less need for fine motor skills to engage a precise locking mechanism with special attachment pieces. Many patients who can benefit from this device include, but are not limited to, spinal cord injury patients who may have some use of upper extremities but limited fine motor skills in their hands. Because the drainage catheter is not “locked in” to the device, there is much less chance of dislodging the device accidentally. Also, if the drain is left in place for continuous drainage, it can simply be secured with tape or a standard securing device the same way SP catheters are secured now. However, if the catheter accidently gets pulled out of the device, this would occur with fairly low resistance through the two valves, and the device (and bladder access) would remain in place. This is a significant benefit for patients, caregivers, and clinicians over a traditional indwelling SP catheter. The non-locking, open-channel system (as well as the device as a whole) are applicable to other indications such as ascites drainage, pleural drainage, or even enteral feeding.

1. A body cavity conduit device comprising:
a proximal end comprising a low-profile first opening positioned external to a subject's body;
a distal end comprising a second opening positioned within a body cavity of the subject; and
a stem comprising a channel that connects the proximal end and the distal end;
wherein at least one of the proximal end and/or the distal end comprises a self-sealing valve.

2. The device of claim 1, wherein the proximal end comprises a self-sealing valve and wherein the distal end comprises a self-sealing valve.

3. The device of claim 1, wherein the self-sealing valve is configured to prevent fluid flow through the stem channel.

4. The device of claim 1, wherein the self-sealing valve comprises a perforation for insertion of an accessory component, wherein the perforation is configured to form a seal around the accessory component when inserted into the stem channel.

5. The device of claim 1, wherein the proximal end comprises a cover cap and plug for insertion into the first opening, wherein the plug is configured to form a seal when inserted into the first opening.

6. The device of claim 1, wherein the low-profile first opening does not extend outwardly more than 5 cm from the subject's body.

7. The device of claim 1, wherein the low-profile first opening extends outwardly from about 1 cm to about 3 cm from the subject's body.

8. The device of claim 1, wherein the proximal end further comprises a retention balloon port and a retention balloon valve.

9. The device of claim 1, wherein the distal end comprises a retention balloon that is positioned within the subject's body cavity.

10. The device of claim 1, wherein the stem comprises a retention balloon channel extending from the retention balloon port to the retention balloon, wherein the retention balloon channel is parallel to the stem channel

11. The device of claim 9, wherein the retention balloon is configured to hold from about 2 ml to about 25 ml of a fluid.

12. The device of claim 1, wherein the stem is no greater than 30 cm in length.

13. The device of claim 1, wherein the stem is from about 1 cm to about 15 cm in length.

14. The device of claim 1, wherein the stem is no greater than 35 Fr in diameter.

15. The device of claim 1, wherein the stem is from about 10 Fr to about 30 Fr in diameter.

16. The device of claim 1, wherein the accessory component comprises at least one of a bladder catheter, a gastrostomy tube, a drainage catheter, an ablation catheter, a catheter for instilling medication or fluid, an endoscope, a needle, and/or a biopsy tool.

17. The device of claim 1, wherein the subject's body cavity comprises at least a portion of a bladder cavity, a stomach cavity, an intestinal cavity, a peritoneal cavity, a pleural cavity, a fluid collection cavity, a retroperitoneal cavity, a kidney cavity or a gall bladder.

18. A kit comprising:
the body cavity conduit device of claim 1; and
an obturator tool, device, balloon and/or dilator.

19. The kit of claim 18, wherein the kit further comprises at least one of the following: a guidewire, a dilator, a stem measuring device, a slip tip syringe, bladder catheter, a gastrostomy tube, a drainage catheter, an ablation catheter, a catheter for instilling medication or fluid, an endoscope, a needle, a biopsy tool, a splitable or separating removable sheath, and a cleaning tool for a channel.

20. A method for inserting the body cavity conduit device of claim 1 into a body cavity of a subject, the method comprising:
insert an obturator tool, device, balloon, and/or dilator positioned at the distal end of the device over a guidewire and into the subject's body cavity such that the distal end of the device is positioned in the subject's body cavity and the proximal end of the device is positioned external to the subject's body;
inflate the retention balloon with a suitable volume of a fluid to secure placement of the device in the subject; and
remove the obturator and the guidewire.
